(19) United States
(12) Patent Application Publication
(10) Pub. No.: US 2010/0152215 A1
(43) Pub. Date: Jun. 17, 2010

(54) METHODS OF IDENTIFYING ACTIVATORS OF LYN KINASE

(75) Inventors: Andrew Reaume, Wayne, PA (US); Michael Saporito, West Chester, PA (US)

Correspondence Address: Pepper Hamilton LLP, 400 Berwyn Park, 899 Cassatt Road, Berwyn, PA, 19312-1183, US

(73) Assignee: MELIOR PHARMACEUTICALS I, INC., Exton, PA (US)

(21) Appl. No.: 12/527,801

(22) PCT Filed: Feb. 20, 2008
(86) PCT No.: PCT/US08/54361

§ 371 Date: Dec. 16, 2009

Related U.S. Application Data
(60) Provisional application No. 60/890,632, filed on Feb. 20, 2007.

(51) Int. Cl. A61K 31/505; C12Q 1/48

(52) U.S. Cl. 514/274; 435/15

(57) ABSTRACT

The present invention relates to methods of identifying activators of lyn kinase by preincubating a test compound in the presence of lyn kinase; adding ATP and substrate to the lyn kinase and test compound; incubating the test compound, lyn kinase, ATP, and substrate; and measuring phosphorylation level of the substrate, whereby an increase in the phosphorylation of the substrate indicates that the test compound is an activator of lyn kinase.

### FIELD OF THE INVENTION

The present invention relates to methods of identifying an activator of lyn kinase and to kits related thereto.

### BACKGROUND OF THE INVENTION

Lyn kinase is a member of the src family of non-receptor protein tyrosine kinases that is predominantly expressed in B-lymphoid and myeloid cells. See, e.g., Briggs S D, Lerner E C, Smithgall T E: Affinity Of Src Family Kinase SH3 Domains For HIV Nef In Vitro Does Not Predict Kinase Activation By Nef In Vivo. Biochemistry 39: 489-495 (2000), incorporated herein by reference. Lyn participates in signal transduction from cell surface receptors that lack intrinsic tyrosine kinase activity. Activation of the lyn kinase activity is necessary for proliferation of CD45+ myeloma cells stimulated by IL-6. See, e.g., Ishikawa H, Tsuyama N, Abroun S, Liu S, Li F J, Taniguchi O, Kawano M M: Requirements of src family kinase activity associated with CD45 for myeloma cell proliferation by interleukin-6 Blood 99: 2172-2178 (2002), incorporated herein by reference. Association of lyn and fyn with the proline-rich domain of glycoprotein VI regulates intracellular signaling. See, e.g., Suzuki-Inoue K, Tulasne D, Shen Y, Bori-Sanz T, Inoue O, Jung S M, Moroi M, Andrews R K, Berndt M C, Watson S P: Association of Fyn and Lyn with the proline-rich domain of glycoprotein VI regulates intracellular signaling. J. Biol. Chem. 277: 21561-21566 (2002), incorporated herein by reference. The lyn/CD22/SHP-1 pathway is important in autoimmunity. See, e.g., Blasioli J, Goodnow C C: Lyn/CD22/SHP-1 and their importance in autoimmunity. Curr. Dir. Autoimmun. 5:151-160 (2002), incorporated herein by reference.

Obesity, hyperlipidemia, and diabetes have been shown to play a causal role in various disorders including, for example, atherosclerotic cardiovascular diseases, which currently account for a considerable proportion of morbidity in Western society. One human disorder, termed “Syndrome X” or “Metabolic Syndrome,” is manifested by defective glucose metabolism (e.g., insulin resistance), elevated blood pressure (i.e., hypertension), and a blood lipid imbalance (i.e., dyslipidemia). See e.g. Reaven, 1993, Annu. Rev. Med. 44:121-131.

None of the currently commercially available drugs for modulating lyn kinase or managing elevated glucose levels have a general utility in regulating lipid, lipoprotein, insulin and glucose levels in the blood. Thus, compounds that have one or more of these utilities are clearly needed. Furthermore, there is a clear need to develop safer drugs that are efficacious at lowering serum cholesterol, increasing HDL serum levels, preventing coronary heart disease, and/or treating existing disease such as atherosclerosis, obesity, diabetes, and other diseases that are affected by glucose metabolism and/or elevated glucose levels. Accordingly, assays for identifying activators of lyn kinase would be greatly desired.

### SUMMARY OF THE INVENTION

The present invention provides methods of identifying an activator of lyn kinase comprising: preincubating a test compound in the presence of lyn kinase; adding ATP and substrate to the lyn kinase and test compound; incubating the test compound, lyn kinase, ATP, and substrate; and measuring the phosphorylation level of the substrate, whereby an increase in the phosphorylation level of the substrate indicates that the test compound is an activator of lyn kinase.

In some embodiments, the test compound is preincubated in the presence of lyn kinase from about 5 minutes to about 120 minutes. In some embodiments, the test compound is preincubated in the presence of lyn kinase from about 30 minutes to about 90 minutes. In some embodiments, the test compound is preincubated in the presence of lyn kinase from about 45 minutes to about 75 minutes. In some embodiments, the test compound is preincubated in the presence of lyn kinase for about 60 minutes. In some embodiments, the test compound is preincubated in the presence of lyn kinase for 20 to 40 minutes.

In some embodiments, the test compound is preincubated in the presence of lyn kinase at about 0° C. to about 30° C. In some embodiments, the test compound is preincubated in the presence of lyn kinase at about 0° C. to about 10° C. In some embodiments, the test compound is preincubated in the presence of lyn kinase at about 4° C.

In some embodiments, the concentration of the lyn kinase is from about 10 ng/ml to about 500 ng/ml. In some embodiments, the concentration of the lyn kinase is from about 25 ng/ml to about 300 ng/ml.

In some embodiments, the concentration of ATP is from about 5 μM about 25 μM. In some embodiments, the concentration of ATP is about 10 μM. In some embodiments, the ATP is radiolabelled.

In some embodiments, the substrate is a protein or peptide that comprises a tyrosine. In some embodiments, the substrate is a synthetic FRET peptide comprising a tyrosine.

In some embodiments, the test compound, lyn kinase, ATP, and substrate are incubated at about room temperature from about 5 minutes to about 90 minutes. In some embodiments, the test compound, lyn kinase, ATP, and substrate are incubated at about room temperature from about 30 minutes to about 75 minutes. In some embodiments, the test compound, lyn kinase, ATP, and substrate are incubated at about room temperature from about 45 minutes to about 60 minutes. In some embodiments, the test compound, lyn kinase, ATP, and substrate are incubated at about room temperature for about 60 minutes. In some embodiments, the test compound, lyn kinase, ATP, and substrate are incubated at room temperature for about 30 to 50 minutes.

In some embodiments, the phosphorylation level of the substrate comprises quantitatively or qualitatively measuring the radiolabelled substrate. In some embodiments, measuring the phosphorylation level of the substrate comprises quantitatively or qualitatively measuring the fluorescence of the synthetic FRET peptide substrate.

In some embodiments, the incubation of the test compound, lyn kinase, ATP, and substrate takes place in the presence of from about 0.05% to about 0.25% bovine serum albumin, from about 0.5 mM to about 2.5 mM dithiothreitol, from about 0.05% to about 0.25% bovine serum albumin and from about 0.5 mM to about 2.5 mM dithiothreitol, or from about 0.05% to about 0.25% β-mercaptoethanol. In some embodiments, the incubation of the test compound, lyn kinase, ATP, and substrate takes place in the presence of from about 0.05% to about 0.25% β-mercaptoethanol. In some embodiments, the incubation of the test compound, lyn kinase, ATP, and substrate takes place in the presence of about 0.1% β-mercaptoethanol.

In some embodiments, the test compound is preincubated in the presence of lyn kinase from about 5 minutes to about 120 minutes; the test compound is preincubated in the presence of lyn kinase at about 0° C. to about 30° C.; and the test compound, lyn kinase, ATP, and substrate are incubated at about room temperature from about 5 minutes to about 90 minutes, in the presence of from about 0.05% to about 0.25% β-mercaptoethanol.

In some embodiments, the test compound is preincubated in the presence of lyn kinase from about 30 minutes to about 90 minutes; the test compound is preincubated in the presence of lyn kinase at about 0° C. to about 10° C.; and the test compound, lyn kinase, ATP, and substrate are incubated at about room temperature from about 30 minutes to about 75 minutes, in the presence of from about 0.05% to about 0.25% β-mercaptoethanol.

In some embodiments, the test compound is preincubated in the presence of lyn kinase from about 45 minutes to about 75 minutes; the test compound is preincubated in the presence of lyn kinase at about 4° C.; and the test compound, lyn kinase, ATP, and substrate are incubated at about room temperature from about 45 minutes to about 60 minutes, in the presence of about 0.1% β-mercaptoethanol.

In some embodiments, the test compound is preincubated in the presence of lyn kinase for about 60 minutes; the test compound is preincubated in the presence of lyn kinase at about 4° C.; and the test compound, lyn kinase, ATP, and substrate are incubated at about room temperature for about 60 minutes, in the presence of about 0.1% β-mercaptoethanol.

In some embodiments, the test compound is a compound of formula II

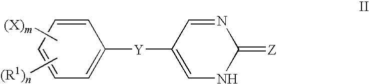

wherein: R¹ is an alkyl group; X is a halogen; Y is O, S, or NH; Z is O or S; n is an integer from 0 to 5 and m is 0 or 1, wherein m+n is less than or equal to 5. In some embodiments, the alkyl group is methyl and n is 1. In some embodiments, the halogen is chlorine and m is 1. In some embodiments, Y is O. In some embodiments, Z is O. In some embodiments, R¹ is methyl, Y is O, Z is O, n is 1, and m is 0. In some embodiments, R¹ is in the meta position. In some embodiments, X is chlorine, Y is O, Z is O, n is 0, and m is 1. In some embodiments, X is in the meta position.

In some embodiments, the test compound is

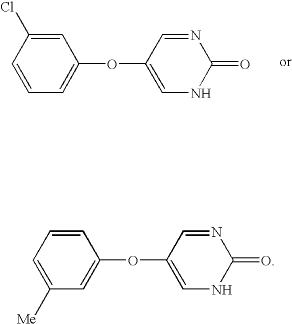

The present invention also provides kits comprising lyn kinase, ATP, substrate, and instructions for carrying out any one or more of the methods described herein. In some embodiments, the kit further comprises an incubation chamber.

The present invention also comprises compositions comprising a first compound of formula I:

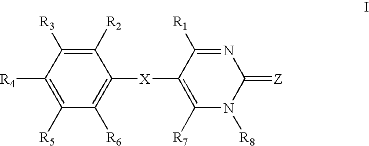

wherein: each of R₁, R₂, R₃, R₄, R₅, R₆, and R₇ are independently a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂; R₈ is alkyl or hydrogen; X is O, S, NH, or N-alkyl; and Z is O or S; or a pharmaceutically acceptable salt thereof; and one or more second compounds, or pharmaceutically acceptable salt thereof, selected from the compounds listed in the Table below. In some embodiments, the first compound is of formula II

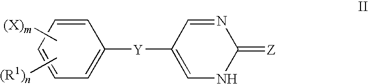

wherein: R¹ is an alkyl group; X is a halogen; Y is O, S, or NH; Z is O or S; and n is an integer from 0 to 5 and m is 0 or 1, wherein m+n is less than or equal to 5.

The present invention also provides methods of treating diabetes in a human comprising administering to the human in need thereof a therapeutically effective amount of a composition described herein.

### BRIEF DESCRIPTION OF THE DRAWINGS

FIG. 1 illustrates synergy of Compound 102 with Metformin.

### DESCRIPTION OF THE INVENTION

As used herein, the term “about” means ±10% of the value it modifies. For example, “about 10” means from 9 to 11.

As used herein, the term “alkoxy” means an —O-alkyl group, wherein alkyl is as defined herein. An alkoxy group can be unsubstituted or substituted with one or two suitable substituents. In some embodiments, the alkyl chain of an alkyloxy group is from 1 to 6 carbon atoms in length, referred to herein, for example, as “(C₁-C₆)alkoxy.”

As used herein, the term “alkenyl” means a monovalent unbranched or branched hydrocarbon chain having one or more double bonds therein. The double bond of an alkenyl group can be unconjugated or conjugated to another unsaturated group. Suitable alkenyl groups include, but are not limited to, (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

As used herein, the term “alkyl” means a saturated, monovalent unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

As used herein, the term “alkynyl” means monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

As used herein, the term “aryl” means a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. In some embodiments, the aryl group is a monocyclic ring, wherein the ring comprises 6 carbon atoms, referred to herein as “(C₆)aryl.”

As used herein, the term “aryloxy” means an —O-aryl group, wherein aryl is as defined herein. An aryloxy group can be unsubstituted or substituted with one or two suitable substituents. In some embodiments, the aryl ring of an aryloxy group is a monocyclic ring, wherein the ring comprises 6 carbon atoms, referred to herein as “(C₆)aryloxy.”

As used herein, the term “benzyl” means —CH₂-phenyl.

As used herein, the term “carbonyl” is a divalent group of the formula —C(O)—.

As used herein, the phrase “compound(s) of the invention” means, collectively, the compounds of formulas I and II, and pharmaceutically acceptable salts thereof. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods. When used in the identification methods described herein, the compounds of the invention are referred to herein as test compounds (which can also serve as positive controls in such methods).

As used herein, the term “cycloalkyl” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. In some embodiments, the cycloalkyl group is a monocyclic ring or bicyclic ring.

As used herein, the term “diabetes” and phrase “type II diabetes” are used interchangeably and include, but are not limited to, non-insulin dependent diabetes mellitus, diabetes insipidus, and are related to insulin resistance (i.e., lack of the ability of the body to respond to insulin appropriately) and is often accompanied by related complications including, for example, obesity and high cholesterol.

As used herein, the term “halogen” means fluorine, chlorine, bromine, or iodine. Correspondingly, the meaning of the terms “halo” and “Hal” encompass fluoro, chloro, bromo, and iodo.

As used herein, the term “heteroaryl” means a monocyclic- or polycyclic aromatic ring comprising carbon atoms, hydrogen atoms, and one or more heteroatoms, such as 1 to 3 heteroatoms, independently selected from nitrogen, oxygen, and sulfur. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, furyl, phenyl, isoxazolyl, and oxazolyl. A heteroaryl group can be unsubstituted or substituted with one or two suitable substituents. In some embodiments, a heteroaryl group is a monocyclic ring, wherein the ring comprises 2 to 5 carbon atoms and 1 to 3 heteroatoms, referred to herein as “(C₂-C₅)heteroaryl.”

As used herein, the term “heterocycloalkyl group” means a monocyclic or polycyclic ring comprising carbon and hydrogen atoms and at least one heteroatom, or 1 to 3 heteroatoms, selected from nitrogen, oxygen, and sulfur, and having no unsaturation. Examples of heterocycloalkyl groups include, but are not limited to, pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. In some embodiments, the heterocycloalkyl group is a monocyclic or bicyclic ring, wherein the ring comprises from 3 to 6 carbon atoms and from 1 to 3 heteroatoms, referred to herein as (C₁-C₆)heterocycloalkyl.

As used herein, the phrase “heterocyclic radical” or “heterocyclic ring” means a heterocycloalkyl group or a heteroaryl group.

As used herein, the term “hydrocarbyl” means a monovalent group selected from (C₁-C₈)alkyl, (C₂-C₈)alkenyl, and (C₂-C₈)alkynyl, optionally substituted with one or two suitable substituents. In some embodiments, the hydrocarbon chain of a hydrocarbyl group is from 1 to 6 carbon atoms in length, referred to herein as “(C₁-C₆)hydrocarbyl.”

As used herein, the term “phenyl” means —C₆H₅. A phenyl group can be unsubstituted or substituted with one or two suitable substituents.

As used herein, the phrase “pre-diabetes” refers to symptoms of diabetes wherein the patient exhibits elevated glucose levels but the full onset of disorders associated with type II diabetes has not yet manifested itself.

As used herein, the phrase “suitable substituent” means a group that does not nullify the synthetic or pharmaceutical utility of the compounds of the invention or the intermediates useful for preparing them. Examples of suitable substituents include, but are not limited to: (C₁-C₈)alkyl; (C₁-C₈)alkenyl; (C₁-C₈)alkynyl; (C₆)aryl; (C₃-C₅)heteroaryl; (C₃-C₇)cycloalkyl; (C₁-C₈)alkoxy; (C₆)aryloxy; —CN; —OH; oxo; halo, —NO₂, —CO₂H; —NH₂; —NH((C₁-C₈)alkyl); —N((C₁-C₈)alkyl)₂; —NH((C₆)aryl); —N((C₆)aryl)₂; —CHO; —CO((C₁-C₈)alkyl); —CO((C₆)aryl); —CO₂((C₁-C₈)alkyl); and —CO₂((C₆)aryl). One of skill in art can readily choose a suitable substituent based on the stability and pharmacological and synthetic activity of the compound of the invention.

As used herein and unless otherwise indicated, the phrase “therapeutically effective amount” of a composition of the invention is measured by the therapeutic effectiveness of a compound of the invention, wherein at least one adverse effect of a disorder is ameliorated or alleviated. In one embodiment, the phrase “therapeutically effective amount” of a composition of the invention is measured by the therapeutic effectiveness of a compound of the invention to alter the expression and/or activity of lyn kinase including, but not limited to up- and down-regulation of this protein. Surprisingly, the inventors have found that therapeutically effective amounts of the compounds of the invention up-regulate the expression and/or activity of lyn kinase.

When used in the methods described herein, the compounds, or pharmaceutically acceptable salt(s) thereof, are used either in isolated form, purified form, or as a mixture of compounds. As used herein, “isolated” means that the compounds are separated from other components of either a natural source, such as a plant or cell, preferably bacterial culture, or a synthetic organic chemical reaction mixture via conventional techniques. As used herein, “purified” means that when isolated, the isolate contains at least 90%, at least 95%, at least 98%, or at least 99% of a compound by weight of the isolate.

The phrase “pharmaceutically acceptable salt(s),” as used herein includes but is not limited to salts of acidic or basic groups that may be present in the compounds used in the present methods. Compounds included in the present methods that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present methods that include an amino moiety may form pharmaceutically acceptable salts with various amino acids, in addition to the acids mentioned above. Compounds, included in the present methods, that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

The present invention provides methods of identifying an activator of lyn kinase comprising: preincubating a test compound in the presence of lyn kinase; adding ATP and substrate to the lyn kinase and test compound; incubating the test compound, lyn kinase, ATP, and substrate; and measuring the phosphorylation level of the substrate, whereby an increase in the phosphorylation level of the substrate indicates that the test compound is an activator of lyn kinase.

The test compound can be any compound that one skilled in the art desires, including any compound described herein. The concentration of the test compound can be any concentration desired and, typically, includes a range of concentrations to be tested.

The lyn kinase can be obtained commercially from, for example, Invitrogen or Millipore, or can be isolated from cells as desired by those skilled in the art. In some embodiments, the concentration of the lyn kinase is from about 10 ng/ml to about 500 ng/ml. In some embodiments, the concentration of the lyn kinase is from about 25 ng/ml to about 300 ng/ml.

In some embodiments, the test compound is preincubated in the presence of lyn kinase from about 5 minutes to about 120 minutes. In some embodiments, the test compound is preincubated in the presence of lyn kinase from about 30 minutes to about 90 minutes. In some embodiments, the test compound is preincubated in the presence of lyn kinase from about 45 minutes to about 75 minutes. In some embodiments, the test compound is preincubated in the presence of lyn kinase for about 60 minutes. In some embodiments, the test compound is preincubated in the presence of lyn kinase for 20 to 40 minutes.

In some embodiments, the test compound is preincubated in the presence of lyn kinase at about 0° C. to about 30° C. In some embodiments, the test compound is preincubated in the presence of lyn kinase at about 0° C. to about 10° C. In some embodiments, the test compound is preincubated in the presence of lyn kinase at about 4° C.

ATP can be commercially obtained from a variety of manufacturers in unlabelled or radiolabelled form. In some embodiments, the concentration of ATP is from about 5 μM to about 25 μM. In some embodiments, the concentration of ATP is about 10 μM.

In some embodiments, the substrate is a protein or peptide that comprises a tyrosine. In some embodiments, the substrate is a synthetic FRET peptide comprising a tyrosine. Numerous FRET peptides comprising a tyrosine are commercially available. One example of a protein or peptide is poly(Glu,Tyr) and can be used at a concentration of from about 0.05 mg/ml to about 0.25 mg/ml. In some embodiments, the concentration of the substrate is about 0.1 mg/ml.

In some embodiments, the test compound, lyn kinase, ATP, and substrate are incubated at about room temperature from about 5 minutes to about 90 minutes. In some embodiments, the test compound, lyn kinase, ATP, and substrate are incubated at about room temperature from about 30 minutes to about 75 minutes. In some embodiments, the test compound, lyn kinase, ATP, and substrate are incubated at about room temperature from about 45 minutes to about 60 minutes. In some embodiments, the test compound, lyn kinase, ATP, and substrate are incubated at about room temperature for about 60 minutes. In some embodiments, the test compound, lyn kinase, ATP, and substrate are incubated at room temperature for about 30 to 50 minutes.

In some embodiments, the phosphorylation level of the substrate comprises quantitatively or qualitatively measuring the radiolabelled substrate. Thus, an increase in phosphorylation of the substrate can be observed without an actual measurement of the amount of phosphorylation. Alternately, the amount of phosphorylation can also be measured by standard techniques known to those skilled in the art. In some embodiments, measuring the phosphorylation level of the substrate comprises quantitatively or qualitatively measuring the fluorescence of the synthetic FRET peptide substrate. Measurement of fluorescence emitted by a FRET peptide is well known to the skilled artisan.

In some embodiments, the incubation of the test compound, lyn kinase, ATP, and substrate takes place in the presence of from about 0.05% to about 0.25% bovine serum albumin, from about 0.5 mM to about 2.5 mM dithiothreitol, from about 0.05% to about 0.25% bovine serum albumin and from about 0.5 mM to about 2.5 mM dithiothreitol, or from about 0.05% to about 0.25% β-mercaptoethanol. In some embodiments, the incubation of the test compound, lyn kinase, ATP, and substrate takes place in the presence of from about 0.05% to about 0.25% β-mercaptoethanol. In some embodiments, the incubation of the test compound, lyn kinase, ATP, and substrate takes place in the presence of about 0.1% β-mercaptoethanol.

In some embodiments, the test compound is preincubated in the presence of lyn kinase from about 5 minutes to about 120 minutes; the test compound is preincubated in the presence of lyn kinase at about 0° C. to about 30° C.; and the test compound, lyn kinase, ATP, and substrate are incubated at about room temperature from about 5 minutes to about 90 minutes, in the presence of from about 0.05% to about 0.25% β-mercaptoethanol.

In some embodiments, the test compound is preincubated in the presence of lyn kinase from about 30 minutes to about 90 minutes; the test compound is preincubated in the presence of lyn kinase at about 0° C. to about 10° C.; and the test compound, lyn kinase, ATP, and substrate are incubated at about room temperature from about 30 minutes to about 75 minutes, in the presence of from about 0.05% to about 0.25% β-mercaptoethanol.

In some embodiments, the test compound is preincubated in the presence of lyn kinase from about 45 minutes to about 75 minutes; the test compound is preincubated in the presence of lyn kinase at about 4° C.; and the test compound, lyn kinase, ATP, and substrate are incubated at about room temperature from about 45 minutes to about 60 minutes, in the presence of about 0.1% β-mercaptoethanol.

In some embodiments, the test compound is preincubated in the presence of lyn kinase for about 60 minutes; the test compound is preincubated in the presence of lyn kinase at about 4° C.; and the test compound, lyn kinase, ATP, and substrate are incubated at about room temperature for about 60 minutes, in the presence of about 0.1% β-mercaptoethanol.

In some embodiments, the test compound is a compound of formula II

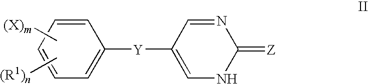

wherein: R¹ is an alkyl group; X is a halogen; Y is O, S, or NH; Z is O or S; n is an integer from 0 to 5 and m is 0 or 1, wherein m+n is less than or equal to 5. In some embodiments, the alkyl group is methyl and n is 1. In some embodiments, the halogen is chlorine and m is 1. In some embodiments, Y is O. In some embodiments, Z is O. In some embodiments, R¹ is methyl, Y is O, Z is O, n is 1, and m is 0. In some embodiments, R¹ is in the meta position. In some embodiments, X is chlorine, Y is O, Z is O, n is 0, and m is 1. In some embodiments, X is in the meta position.

In some embodiments, the test compound is

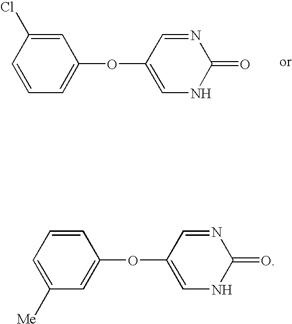

The present invention also provides kits comprising lyn kinase, ATP, substrate, and instructions for carrying out any one or more of the methods described herein. In some embodiments, the kit further comprises an incubation chamber. Incubation chambers, such as microtiter plates, microcentrifuge tubes, and the like are well known to those skilled in the art.

The present invention also comprises compositions comprising a first compound of formula I:

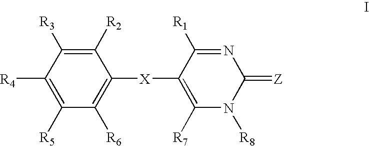

wherein:

each of R₁, R₂, R₃, R₄, R₅, R₆, and R₇ are independently a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂;

R₈ is alkyl or hydrogen;

X is O, S, NH, or N-alkyl; and

Z is O or S; or a pharmaceutically acceptable salt thereof; and one or more second compounds, or pharmaceutically acceptable salt thereof, selected from the compounds listed in Table I.

In some embodiments, R₈ is alkyl, such as methyl. In some embodiments, R₈ is hydrogen.

In some embodiments, X is oxygen.

In some embodiments, Z is oxygen.

In some embodiments, at least one of R₂-R₆ is alkyl, such as methyl. In some embodiments, at least one of R₂-R₆ is halogen, such as chloro. In some embodiments, at least one of R₂-R₆ is —CN. In some embodiments, at least one of R₂-R₆ is —OH. In some embodiments, at least one of R₂-R₆ is —NO₂. In some embodiments, at least one of R₂-R₆ is —CF₃. In some embodiments, at least one of R₂-R₆ is —CO₂H. In some embodiments, at least one of R₂-R₆ is —NH₂. In some embodiments, at least one of R₂-R₆ is -alkoxy.

In some embodiments, R₂ is alkyl, such as methyl and each of R₁, and R₃-R₈ is hydrogen and X and Z are O.

In some embodiments, R₂ is a halogen, such as chloro, and each of R₁, and R₃-R₈ is hydrogen and X and Z are O.

In some embodiments, R₃ is alkyl, such as methyl, and each of R₁, R₂ and R₄-R₈ is hydrogen and X and Z are O.

In some embodiments, R₃ is a halogen, such as chloro, and each of R₁, R₂, and R₄-R₈ is hydrogen and X and Z are O.

In some embodiments, R₄ is alkyl, such as methyl, and each of R₁-R₃ and R₅-R₈ is hydrogen and X and Z are O.

In some embodiments, R₄ is a halogen, such as chloro, and each of R₁-R₃, and R₅-R₈ is hydrogen and X and Z are O.

In some embodiments, R₅ is —CF₃, and each of R₁-R₄ and R₆-R₈ is hydrogen and X and Z are O.

In some embodiments, R₅—NH₂, and each of R₁-R₄ and R₆-R₈ is hydrogen and X and Z are O.

In some embodiments, R₆ is —CF₃, and each of R₁-R₅ and R₇-R₈ is hydrogen and X and Z are O.

In some embodiments, R₆ is —NH₂ and each of R₁-R₅ and R₇-R₈ is hydrogen and X and Z are O.

In some embodiments, the first compound is of formula II

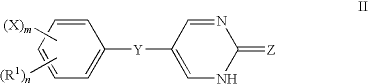

wherein:

R¹ is an alkyl group;

X is a halogen;

Y is O, S, or NH;

Z is O or S; and

n is an integer from 0 to 5 and m is 0 or 1, wherein m+n is less than or equal to 5.

Illustrative examples of compounds that are useful in the compositions and methods described herein include, but are not limited to:

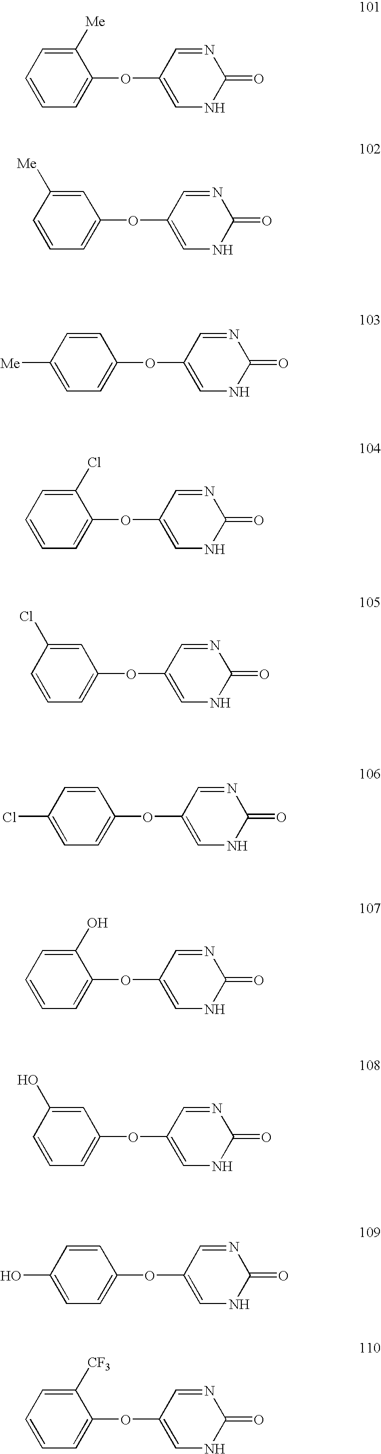

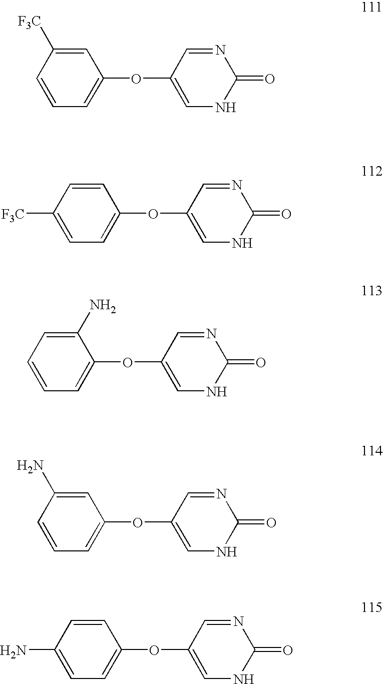

The compounds can be synthesized by organic chemistry techniques known to those of ordinary skill in the art, for example as described in U.S. Pat. No. 3,922,345, which is incorporated herein by reference in its entirety.

The present invention also provides methods of treating diabetes in a human comprising administering to the human in need thereof a therapeutically effective amount of a composition described herein.

In some embodiments of the invention, the compounds of the invention can be used in combination therapy with at least one other therapeutic agent. The compound of the invention and the therapeutic agent can act additively or synergistically. In one embodiment, a composition comprising a compound of the invention is administered concurrently with the administration of another therapeutic agent, which can be part of the same composition as the compound of the invention or a different composition. In another embodiment, a composition comprising a compound of the invention is administered prior or subsequent to administration of another therapeutic agent. In one embodiment, combination therapy involves alternating between administering a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In some embodiments, when a composition of the invention is administered concurrently with another therapeutic agent that potentially produces adverse side effects including, but not limited to, toxicity, the therapeutic agent can advantageously be administered at a dose that falls below the threshold at which the adverse side is elicited.

In one embodiment, “treatment” or “treating” refers to an amelioration of diabetes, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of diabetes, either physically, e.g., stabilization of a discernible symptom, physiologically, e.g., stabilization of a physical parameter, or both. In yet another embodiment, “treatment” or “treating” refers to delaying the onset of diabetes.

In some embodiments, the compositions of the invention are administered to a patient, preferably a human, as a preventative measure against diabetes. As used herein, “prevention” or “preventing” refers to a reduction of the risk of acquiring diabetes. In one embodiment, the compositions of the present invention are administered as a preventative measure to a patient, preferably a human having a genetic predisposition to diabetes. In another embodiment, the compositions of the invention are administered as a preventative measure to a subject having a non-genetic predisposition to diabetes.

As used herein, “treatment or prevention of diabetes” encompasses treatment or prevention of a complication associated with type II diabetes including, but not limited to, retinopathy (i.e., blindness); neuropathy (i.e., nerve damage) which leads to foot ulcers, gangrene, and amputations; kidney damage, which leads to dialysis; and cardiovascular disease. In some embodiments, the type II diabetes is associated with abnormal/altered lyn kinase activity and/or expression.

The compositions comprising a compound of the invention are therefore useful in treating or preventing type II diabetes or complications arising from type II diabetes and disorders and risk factors associated with metabolic syndrome. Complications of diabetes include, but are not limited to, diabetic neuropathy, diabetic retinopathy, erectile dysfunction, and kidney disease and the compounds of the invention are useful in treating or preventing these complications.

The invention provides methods of treatment and prophylaxis by administration to a patient of a therapeutically effective amount of a composition comprising a compound of the invention. The patient is a mammal, including, but not limited, to an animal such a cow, horse, sheep, pig, chicken, turkey, quail, cat, dog, mouse, rat, rabbit, guinea pig, etc., and is more preferably a human.

The present compositions, which comprise one or more compounds of the invention, can be administered orally. The compounds of the invention can also be administered by any other convenient route, for example, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In some embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner, and will depend in-part upon the site of the medical condition. In most instances, administration will result in the release of the compounds of the invention into the bloodstream.

In some embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In some embodiments, the compounds of the invention can be formulated as a suppository, with traditional binders and vehicles such as triglycerides.

In another embodiment, the compounds of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compounds of the invention, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a compound of the invention, optionally more than one compound of the invention, suitably in purified form, together with a suitable amount of a pharmaceutically acceptable vehicle so as to provide the form for proper administration to the patient.

In some embodiments, the phrase “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, A. R. Gennaro (Editor) Mack Publishing Co.

In one embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. Where necessary, the compositions may also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

The compositions of the invention be administered orally. Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are suitably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In some embodiments, the oral dose is 0.01 milligram to 70 milligrams per kilogram body weight, or 0.1 milligram to 50 milligrams per kilogram body weight, or 0.5 milligram to 20 milligrams per kilogram body weight, or 1 milligram to 10 milligrams per kilogram body weight. In some embodiments, the oral dose is 5 milligrams of a compound of the invention per kilogram body weight. The dosage amounts described herein refer to total amounts administered; that is, if more than one compound of the invention is administered, the dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The table below describes drug targets for Type II diabetes with representative drugs (approved for use), development stage drugs, or preclinical compounds to that target, any one or more of which can be used in combination with any one or more of the compounds of the present invention.

TABLE I

| Drug or Compound | | |
|---|---|---|
| Name | CAS Number | MECHANISM |
| | 859721-77-0 | 11-beta-Hydroxysteroid Dehydrogenase Type 1 |
| | | Inhibitor |
| LG-101506 | 331248-11-4 | ABCA1 Expression Enhancer Retinoid RXR |
| | | Modulator |
| | 232252-56-1 | Adenosine A2B Antagonist |
| Acarbose | 056180-94-0 | alpha-Glucosidase Inhibitors |
| A-769662 | 844499-71-4 | AMP-Activated Protein Kinase (AMPK) |
| | | Activator |
| NVP-DPP-728, | 207556-62-5 | DPP-IV inhibitor |
| sitagliptin | | |
| MB-05032 | 261365-11-1 | Fructose-1,6-Bisphosphatase Inhibitor |
| | 886451-10-1 | G Protein-Coupled Receptor GPR40 Agonist |
| | 882509-86-6 | G Protein-Coupled Receptor GPR40 Antagonist |
| GLP-1 or GLP-1 | | GLP-1 Receptor Agonist |
| fragment | | |
| BAY-27-9955 | 202855-56-9 | Glucagon antagonist |
| LY-2121260 | 731018-97-6 | Glucokinase activator |
| | 260545-12-8 | Glucose-6-phosphatase Inhibitor |
| BAY-R-3401 | 100276-03-7 | Glycogen Phosphorylase Inhibitor |
| | 890050-91-6 | Glycogen Synthase Activator |
| SB-216763 | 280744-09-4 | GSK-3 Inhibitor |
| Leporin B | 175883-72-4 | HK2 Expression Enhancer |
| TER-16998 | | Insulin sensitizer |
| CRx-401 Bezafibrate/ | | Insulin sensitizer |
| diflunisal | | |
| | 325979-95-1 | IRK activator |
| Tolbutamide | 000064-77-7 | K(ATP) Channel Blockers |
| | 748166-36-1 | Melanin-Concentrating Hormone MCH |
| | | Receptor Antagonist |
| | | Melanocortin MC4 Agonist |
| SB-334867-A | | Orexin OX-1 Antagonist |
| | 748147-23-1 | Phosphoenolpyruvate Carboxykinase (PEPCK) |
| | | Inhibitor |
| | | PPARalpha Agonist PPARgamma Agonist |
| Rosiglitazone | 155141-29-0 | PPARgamma Agonist |
| | 737805-90-2 | PPARgamma Partial Agonist |
| Diethyl lutidinate | 041438-38-4 | Prolyl 4-Hydroxylase Inhibitor |
| | 251475-05-5 | PTP 1b inhibitor |
| Sergliflozin | | SGLT-2 Inhibitors |
| | 916338-41-5 | Somatostatin SRIF1B (sst5) Antagonist |
| | 916888-66-9 | Stearoyl-CoA 9 Desaturase Inhibitor |
| | 811811-95-7 | Triacylglycerol Lipase Inhibitor |
| Metformin | 001115-70-4 | Unknown |
| DecbSM | 148565-56-4 | Unknown |
| NNC-57-0511 | | Unknown |

The present invention is also directed to the use of the compositions described herein for the treatment of diabetes.

The present invention is also directed to use of the compounds and/or compositions described herein in the preparation of a medicament for use in the treatment of diabetes.

In order that the invention disclosed herein may be more efficiently understood, examples are provided below. It should be understood that these examples are for illustrative purposes only and are not to be construed as limiting the invention in any manner.

### EXAMPLES

### Example 1

### Lyn Kinase Selectivity

A kinase screen was conducted in which Compound 102 (10 μM) was screened against 49 kinases for inhibition or activation. Any kinase that was activated or inhibited by at least 20% was considered to be a significant effect. Of the 49 kinases screened, Lyn kinase was activated by 51%. In a repeat experiment, Lyn kinase activity was increased by 57%. Of the other kinases tested, insulin-like growth factor receptor activity was inhibited by 20.1%, and p70S6K was inhibited by 24.3%.

| | COMPOUND 102 ACTIVITY |
|---|---|
| KINASE | (% KINASE ACTIVITY) |
| AMPK | −0.95 |
| Abl | −4.04 |
| Akt1 | 1.51 |
| Akt2 | −6.83 |
| AurA | −12.21 |
| BTK | 10 |
| CDK2/cyclinA | −3.93 |
| CHK1 | 0.87 |
| CHK2 | −6.17 |
| CK1d | −1.5 |
| CaMK2a | 0.08 |
| CaMK4 | 17.35 |
| EphA2 | 1.03 |
| FGFR3 | −1.58 |
| Flt3 | 7.34 |
| Fyn | −4.64 |
| GSK3b | 2.47 |
| IGF1R | −20.18 |
| IKKb | −6.26 |
| IRAK4 | 2.25 |
| InsR | 16.92 |
| JNK2 | −4.8 |
| KDR (FLK1, VEGFR2) | 0 |
| Lck | 6.28 |
| Lyn | 51.05 |
| Lyn (repeat) | 57.11 |
| Lyn | 57 |
| MAPK1 | 0.6 |
| MAPK14 (P38) | 1.74 |
| MAPK3 (p38) | −6.05 |
| MAPKAP2 | −13.04 |
| MAPKAP5 | 0.79 |
| MET | 9.88 |
| MSK1 | 1.55 |
| MST2 | 4.59 |
| PAK2 | 1.55 |
| PDK1 | −2.63 |
| PKA | −0.3 |
| PKCb2 | −1.12 |
| PKCz | 9.05 |
| PKD2 | −10.34 |
| Pim2 | −8.06 |
| ROCK2 | −3.05 |
| RSK1 | 1.72 |
| SGK1 | −19.24 |
| Src | 4.56 |
| Syc | 11.12 |
| cKit | 18.79 |
| cRAF | 5.08 |

### Example 2

### Lyn Kinase Activation Optimization and ATP Kinetics

After identification of the Lyn kinase activation, studies were repeated to optimize the conditions of Compound 102-mediated Lyn kinase activation. The following studies were conducted: 1) optimize the pre-incubation conditions for maximizing Compound 102-mediated activation of Lyn; and 2) testing to determine the enzyme kinetics mediating Compound 102 activation of Lyn.

### Optimal Preincubation Conditions

In the experiment, the kinase Lyn (h) was pre-incubated with DMSO or 1, 3, 10, 30, and 100 μM of Compound 102 for 30 minutes on ice. The kinase was then diluted to a concentration that gives linear kinetics and assayed in a standard radiometric assay at 10 μM ATP for 40 minutes at room temperature.

In this experiment, the activity of Lyn kinase (h) increased with Compound 102 concentration in a dose-dependent manner. At 100 μM of Compound 102, the activity of Lyn kinase (h) was more than 3-fold greater than the activity measured in the DMSO control. The EC50 for Compound 102 activation of Lyn kinase was 650 nM.

### ATP Kinetic Study

The kinetic study was designed using the preincubation and experimental conditions established above. In this study, the Compound 102 concentration was held constant at 100 μM and the ATP concentration increased stepwise from 0 to 800 μM. Kinetic parameters for ATP were calculated using non-linear regression analysis and the equation: Y=Vmax(x)/(Km+x). Vmax=the activity of the enzyme and Km=the concentration of ATP needed for half-maximal activity. Under these conditions, Compound 102 increased the Vmax of Lyn kinase by 3-fold but did not alter the Km for ATP. Thus, it appears that Compound 102 increases Lyn kinase in an ATP-independent manner.

### Example 3

### Screening for Lyn Activation

A Lyn kinase activation high throughput screen is presently being established using conditions established in the optimization assays described in Example 2. This assay will be used to identify new activators of Lyn kinase. These conditions are presented here: 1) preincubation of test compound in the presence of Lyn kinase for 30 minutes at 4° C.; 2) assaying initiated by addition of ATP and substrate; 3) incubation at room temperature for about 40 minutes; and 4) measuring phosphorylation level of the substrate.

### Example 4

### Kinase Activation In Vivo

CD1 male mice 8 weeks of age were used for these studies. Mice were maintained with free access to food and water and kept on a 12 hour light/dark cycle.

Mice were fasted for 18 hours and baseline blood glucose measured. Sixty minutes after baseline glucose levels, mice were dosed with vehicle, 30 or 100 mg/kg of Compound 102. Livers were dissected free 75 minutes after drug administration.

Livers were homogenized using a bead-beater and 1.0 mm glass beads in 0.75 ml lysis buffer (50 mM Tris-HCl, 1% NP-40, 0.25% Na-deoxycholate, 150 mM NaCl, 1 mM EDTA, 1 mM PMSF, 1 μg/ml Aprotinin, 1 μg/ml Leupeptin, 1 mM Na₃VO₄, 1 mM NaF, 10% Glycerol, pH 7.4). Tissue homogenates were centrifuged and the supernatant tested for levels of phosphor-IRS-1 (see below).

Phospho-tyrosine tagged proteins were immunoprecipitated using a monoclonal phospho-tyrosine antibody and Protein Sepharose A. Immunoprecipitated proteins were separated using a PAGE electrophoresis system (Invitrogen; NuPage system). Proteins were transferred to PVDF membranes. Phosphorylated IRS-1 was detected on the PVDF membranes using a pan-IRS-1 rabbit polyclonal antibody, secondary-HRP chemiluminescence (Upstate).

### Example 5

### Lyn Kinase Activation Assay Optimization

The following example was conducted to further optimize assay conditions for detecting Compound 102-mediated activation of Lyn kinase. The assays describe below utilize the Z′-LYTE™ Kinase Assay Kit-Tyr Peptide (Invitrogen, Carlsbad, Calif.). These assay systems were originally developed to detect inhibitors of various tyrosine kinases. The modifications that have been added to the system optimize conditions for detecting activators of lyn kinase. Specifically, the optimal Lyn kinase protein concentrations, buffer components, and assay conditions for detecting Compound 102-mediated activation of Lyn kinase are described herein.

The assay system used was a non-radiometric assay utilizing the Z′-LYTE™ Kinase Assay Kit-Tyr Peptide-2 (Invitrogen, Carlsbad, Calif.) for detecting activity of Lyn kinase. Buffer components were altered as described below. The lyn kinase protein used in these studies was the unphosphorylated form of the protein. Concentrations of this unphosphorylated lyn protein ranged from 25 ng/mL to 300 ng/mL. The preincubation step was lengthened from 30 minutes to 60 minutes.

The Invitrogen Reaction Buffer was used for all studies. The following additions were made to this Reaction Buffer and tested separately and compared to Reaction Buffer alone: Modification 1: Reaction Buffer with 0.1% BSA; Modification 2: Reaction Buffer with 1 mM dithiothreitol; Modification 3: Reaction Buffer with 0.01% BSA, 1 mM dithiothreitol; and Modification 4: Reaction Buffer with 0.1% β-mercaptoethanol.

Table II describes the different activation levels with the different buffer components.

TABLE II
Compound 102-Mediated Activation of Lyn Kinase

| | DMSO | Compound 102 | |
|---|---|---|---|
| | (control); % | (30 μM); % | |
| | Phosphorylation | Phosphorylation | Fold- |
| Buffer | of Substrate | of Substrate | Activation |
| Reaction Buffer | 10.4 | 16.2 | 1.6 |
| Modification 1 | 30.8 | 33.6 | 1.1 |
| Modification 2 | 67.1 | 80.9 | 1.2 |
| Modification 3 | 51.7 | 83.4 | 1.6 |
| Modification 4 | 13.7 | 33.3 | 2.4 |

Buffer Modification 4 produced the optimal activation conditions for Lyn kinase. Addition of 0.1% β-mercaptoethanol to the Reaction Buffer (modification 4) resulted in a low baseline level (13.7% substrate phosphorylation) and, with addition of Compound 102, a 2.4 fold activation of Lyn kinase. Modification 4 buffer produced significantly better activation than Modifications 1, 2 and 3.

These studies describe the optimal, consistent, conditions for detecting Compound 102-mediated activation of Lyn kinase. These optimal conditions are as follows: 1) utilization of the T-LYTE™ Kinase Assay Kit-Tyr Peptide-2 (Invitrogen, Carlsbad, Calif.); 2) utilization of unphosphorylated Lyn kinase protein at concentrations ranging from 25 ng/mL to 300 ng/mL; 3) standard reaction buffer from Invitrogen that is supplemented with 0.1% β-mercaptoethanol; and 4) lengthening the pre-incubation step from 30 to 60 minutes in the absence of ATP.

### Example 6

### Combination Therapies

Based on preliminary data, Compound 102 will be useful in treatment in combination with existing Type II diabetic agents. Animals were treated separately and in combination at non-effective doses. Independently, drugs did not affect blood glucose levels in an oral glucose tolerance test. In combination, drugs significantly decreased blood glucose levels. In addition, studies that are ongoing include Compound 102 in combination with a sulfonylurea (glybenclamide) and Compound 102 in combination with rosiglitazone in db/db mice.

CD1 male mice were fasted for 18 hrs. After fast, animals were treated with drug at the indicated doses (see Tables below).

| TIME | EVENT |
|---|---|
| −18 hrs | Fast |
| 0 | Measure glucose |
| +60 | Drug Administration |
| +75 | Glucose challenge |
| +135 | Measure glucose |

| TREATMENT | N |
|---|---|
| Vehicle Control | 6 |
| Metformin (200 mg/kg) | 6 |
| Compound 102 (30 mg/kg) | 6 |
| Metformin (200 mg/kg) | 6 |
| Compound 102 (30 mg/kg) | |
| No Glucose/No drug | 4 |

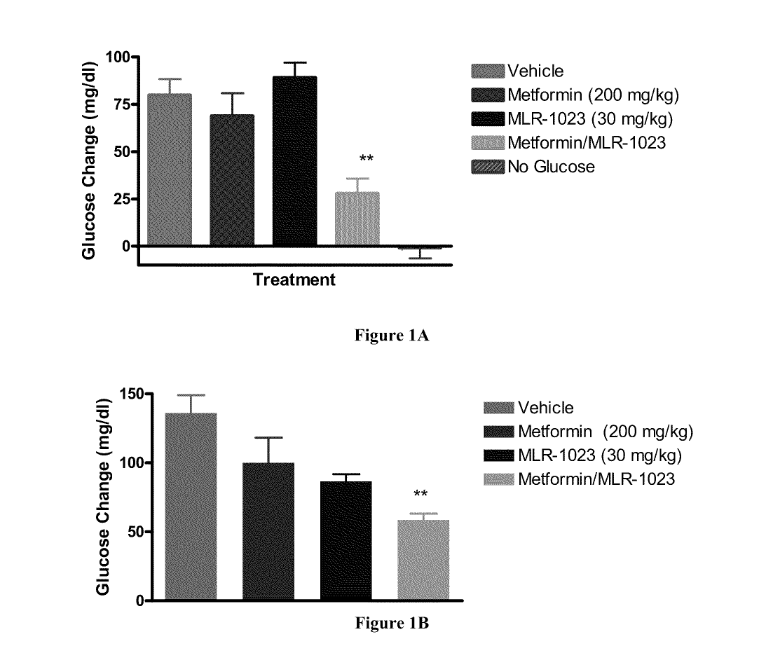

Synergy of Compound with Metformin is demonstrated in FIG. 1.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety. U.S. Ser. No. 60/890,632 filed Feb. 20, 2007 is incorporated herein by reference in its entirety.

1. A method of identifying an activator of lyn kinase comprising:
preincubating a test compound in the presence of lyn kinase;
adding ATP and substrate to the lyn kinase and test compound;
incubating the test compound, lyn kinase, ATP, and substrate; and
measuring the phosphorylation level of the substrate, whereby an increase in the phosphorylation level of the substrate indicates that the test compound is an activator of lyn kinase.

2. The method of claim 1 wherein the test compound is preincubated in the presence of lyn kinase from about 5 minutes to about 120 minutes.

3-5. (canceled)

6. The method of claim 1 wherein the test compound is preincubated in the presence of lyn kinase at about 0° C. to about 30° C.

7-8. (canceled)

9. The method of claim 1 wherein the concentration of the lyn kinase is from about 10 ng/ml to about 500 ng/ml.

10. (canceled)

11. The method of claim 1 wherein the concentration of ATP is from about 5 μM to about 25 μM.

12. (canceled)

13. The method of claim 1 wherein the ATP is radiolabelled.

14. The method of claim 1 wherein the substrate is a protein or peptide that comprises a tyrosine.

15. The method of claim 1 wherein the substrate is a synthetic FRET peptide comprising a tyrosine.

16. The method of claim 1 wherein the test compound, lyn kinase, ATP, and substrate are incubated at about room temperature from about 5 minutes to about 90 minutes.

17-19. (canceled)

20. The method of claim 13 wherein measuring the phosphorylation level of the substrate comprises quantitatively or qualitatively measuring the radiolabelled substrate.

21. The method of claim 15 wherein measuring the phosphorylation level of the substrate comprises quantitatively or qualitatively measuring the fluorescence of the synthetic FRET peptide substrate.

22. The method of claim 1 wherein the incubation of the test compound, lyn kinase, ATP, and substrate takes place in the presence of from about 0.05% to about 0.25% bovine serum albumin, from about 0.5 mM to about 2.5 mM dithiothreitol, from about 0.05% to about 0.25% bovine serum albumin and from about 0.5 mM to about 2.5 mM dithiothreitol, or from about 0.05% to about 0.25% β-mercaptoethanol.

23. The method of claim 1 wherein the incubation of the test compound, lyn kinase, ATP, and substrate takes place in the presence of from about 0.05% to about 0.25% β-mercaptoethanol.

24. The method of claim 1 wherein the incubation of the test compound, lyn kinase, ATP, and substrate takes place in the presence of about 0.1% β-mercaptoethanol.

25. The method of claim 1 wherein:
the test compound is preincubated in the presence of lyn kinase from about 5 minutes to about 120 minutes;
the test compound is preincubated in the presence of lyn kinase at about 0° C. to about 30° C.; and
the test compound, lyn kinase, ATP, and substrate are incubated at about room temperature from about 5 minutes to about 90 minutes, in the presence of from about 0.05% to about 0.25% β-mercaptoethanol.

26-28. (canceled)

29. The method of claim 1 wherein the test compound is a compound of formula II 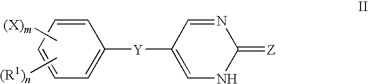
wherein:
R¹ is an alkyl group;
X is a halogen;
Y is O, S, or NH;
Z is O or S;
n is an integer from 0 to 5 and m is 0 or 1, wherein m+n is less than or equal to 5.

30-37. (canceled)

38. The method of claim 29 wherein the test compound is

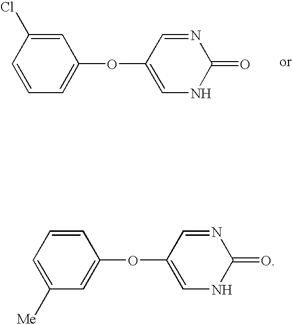

39. A kit comprising lyn kinase, ATP, substrate, and instructions for carrying out the method of claim 1.

40. (canceled)

41. A composition comprising a first compound of formula II: 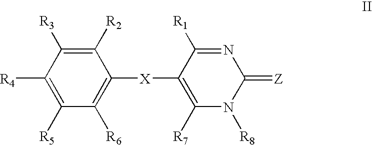
wherein
each of R₁, R₂, R₃, R₄, R₅, R₆, and R₇ are independently a hydrogen, alkoxy, alkyl, alkenyl, alkynyl, aryl, aryloxy, benzyl, cycloalkyl, halogen, heteroaryl, heterocycloalkyl, —CN, —OH, —NO₂, —CF₃, —CO₂H, —CO₂alkyl, or —NH₂;
R₈ is alkyl or hydrogen;
X is O, S, NH, or N-alkyl; and
Z is O or S; or a pharmaceutically acceptable salt thereof; and
one or more second compounds, or pharmaceutically acceptable salt thereof, selected from the compounds listed in Table I.

42. The composition of claim 41 wherein the first compound is of formula II 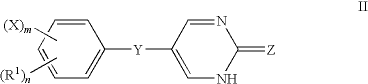
wherein:
R¹ is an alkyl group;
X is a halogen;
Y is O, S, or NH;
Z is O or S; and
n is an integer from 0 to 5 and m is 0 or 1, wherein m+n is less than or equal to 5.

43. A method of treating diabetes in a human comprising administering to the human in need thereof a therapeutically effective amount of a composition of claim 41.

44. A pharmaceutical composition comprising a compound of formula II 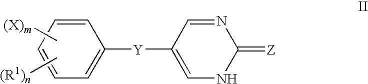
wherein:
R¹ is an alkyl group;
X is a halogen;
Y is O, S, or NH;
Z is O or S; and
n is an integer from 0 to 5 and m is 0 or 1, wherein m+n is less than or equal to 5, or a pharmaceutically acceptable salt thereof; and
rosiglitazone.

45. The pharmaceutical composition of claim 44 wherein the compound of formula II is

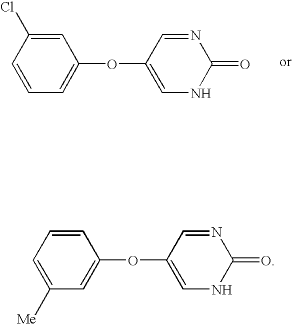

46. The pharmaceutical composition of claim 44 wherein the compound of formula II is [chemical structure]
